(19) United States
(12) Patent Application Publication
(10) Pub. No.: US 2008/0076836 A1
(43) Pub. Date: Mar. 27, 2008

(54) METHOD AND APPARATUS FOR USING LIGHT TO ENHANCE CELL GROWTH AND SURVIVAL

(75) Inventors: Darrell O. Wagner, Isanti, MN (US); Haris J. Sih, Minneapolis, MN (US); Steven D. Girouard, Chagrin Falls, OH (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc, st paul, MN (US)

(21) Appl. No.: 11/843,573

(22) Filed: Aug. 22, 2007

Related U.S. Application Data
(60) Provisional application No. 60/842,201, filed on Sep. 1, 2006.

(51) Int. Cl. A61K 31/00; A61P 43/00; C12P 1/00; C12Q 1/02

(52) U.S. Cl. 514/789; 435/29; 435/41

(57) ABSTRACT

A biologic agent preparation system is provided for producing one or more biologic agents in vitro including pro-growth factors and/or pro-survival factors from cells. The one or more biologic agents are produced in response to exposure of mammalian cells to light. A biologic agent delivery system is also provided that delivers the pro-growth factors and/or pro-survival factors to an injured tissue region, or delivers cells treated with the pro-growth factors and/or pro-survival factors to the injured tissue region.

### CROSS-REFERENCE TO RELATED APPLICATIONS

This application claims the benefit of U.S. Provisional Application Ser. No. 60/842,201, filed Sep. 1, 2006 under 35 U.S.C. § 119(e) which is incorporated herein by reference in its entirety.

This application is related to co-pending, commonly assigned, U.S. Patent Application Ser. No. 60/824,381, entitled “METHOD AND APPARATUS UTILIZING LIGHT AS THERAPY FOR FUNGAL INFECTION,” filed on Sep. 1, 2006 and U.S. patent application Ser. No. 11/469,767, entitled “METHODS AND SYSTEMS FOR TREATING POST-MI PATIENTS,” filed on Sep. 1, 2006, which are hereby incorporated herein by reference in their entirety.

### TECHNICAL FIELD

This document relates generally to medical devices and particularly to a system that uses light to induce production of one or more biologic agents that enhance cell growth and survival.

### BACKGROUND

The heart is the center of a person's circulatory system. It includes an electro-mechanical system performing two major pumping functions. The heart includes four chambers: right atrium (RA), right ventricle (RV), left atrium (LA), and left ventricle (LV). The left portions of the heart, including LA and LV, draw oxygenated blood from the lungs and pump it to the organs of the body to provide the organs with their metabolic needs for oxygen. The right portions of the heart, including RA and RV, draw deoxygenated blood from the body organs and pump it to the lungs where the blood gets oxygenated. The efficiency of the pumping functions, indicative whether the heart is normal and healthy, is indicated by measures of hemodynamic performance, such as parameters related to intracardiac blood pressures and cardiac output.

In a normal heart, the sinoatrial node, the heart's natural pacemaker, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract in synchrony to result in efficient pumping functions indicated by a normal hemodynamic performance. A blocked or otherwise abnormal electrical conduction and/or deteriorated myocardial tissue cause dysynchronous contraction of the heart, resulting in poor hemodynamic performance, including a diminished blood supply to the heart and the rest of the body. The condition where the heart fails to pump enough blood to meet the body's metabolic needs is known as heart failure.

Myocardial infarction (MI) is the necrosis of portions of the myocardial tissue resulted from cardiac ischemia, a condition in which the myocardium is deprived of adequate oxygen and metabolite removal due to an interruption in blood supply. The adult heart lacks a substantial population of precursor, stem cells, or regenerative cells. Therefore, after MI, the heart lacks the ability to effectively regenerate cardiomyocytes to replace the injured cells in the infarcted areas of the myocardium. Each injured area eventually becomes a fibrous scar that is non-conductive and non-contractile. Consequently, the overall contractility of the myocardium is weakened, resulting in decreased cardiac output. As a physiological compensatory mechanism that acts to increase cardiac output in response to MI, the LV diastolic filling pressure increases as the pulmonary and venous blood volume increases. This increases the LV preload (stress on the LV wall before its contracts to eject blood). One consequence is the progressive change of the LV shape and size, a processes referred to as remodeling. Remodeling is initiated in response to a redistribution of cardiac stress and strain caused by the impairment of contractile function in the infarcted tissue as well as in nearby and/or interspersed viable myocardial tissue with lessened contractility due to the infarct. The remodeling starts with expansion of the region of the infarcted tissue and progresses to a chronic, global expansion in the size and change in the shape of the entire LV. Although the process is initiated by the compensatory mechanism that increases cardiac output, the remodeling ultimately leads to further deterioration and dysfunction of the myocardium. Consequently, post MI patients experience impaired hemodynamic performance and have a significantly increased risk of developing heart failure.

Therefore, there is a need for treating myocardial injuries after MI.

### SUMMARY

An in vitro biologic agent preparation system produces a composition which includes one or more biologic agents including pro-growth factors and/or pro-survival factors. The composition is prepared by exposing cells in vitro, e.g., mammalian cells in media in vitro, to a wavelength of light or a band of wavelengths of light, which results in increased production of pro-growth factors and/or pro-survival factors by the cells. Those factors are released, e.g., secreted, from the cells, for example, into the media, and isolated. A biologic agent delivery system delivers the composition to an injured tissue region or delivers cells treated in vitro with the composition to an injured tissue region.

In one embodiment, the invention provides a method to identify a wavelength of light that enhances expression of pro-growth factors and/or pro-survival factors. The method includes contacting in vitro a first population of mammalian cells in culture media with a wavelength of light or a band of wavelengths of light. The resulting media is collected and at least a portion is added to a second population of mammalian cells which is different than the first population of cells. Then it is determined whether the media enhances survival or growth of the second population of cells.

The invention also provides for a method of utilizing light, such as red and/or infrared light, to induce cells to express pro-growth factors and/or pro-survival factors in vitro. Cells useful in the method are light-responsive cells, such as fibroblast cells, that produce pro-growth factors or pro-survival factors in an increased amount after light exposure. The preferred light for exposure includes, but is not limited to, red light at a wavelength within a range of 600 nm to 720 nm. Red light and other light having wavelengths in the range of 720 nm to 1000 nm (infrared) may also be used separately or in combination. The factors, which are released from cells exposed to light, are isolated and optionally purified. These factors may then be administered to damaged organs of a mammal, such as the heart, to enhance cell growth and survival, thereby aiding in the natural healing process. For instance, the factors may be implanted into a region of injury within the body to promote cell growth and survival of endogenous cells, including recruited endogenous cells, or implanted (donor) cells such as stem cells.

In one embodiment, an isolated composition comprising the factors is provided in dry (lyophilized) form that may be injected or combined with a pharmaceutically acceptable carrier and injected into a myocardial infarction (MI) or other injured region of the heart or other organs of the body. In another embodiment, the composition may be administered to a blood vessel, e.g., an artery upstream of the damaged organ. The composition may also be applied to a catheter, e.g., one that is acutely placed, or a lead, e.g., one that is chronically implanted, and the factors therein allowed to elute over time in the region of an injury in the heart or other organ. In another embodiment, the composition is applied as a coating on a stent. Stents are commonly placed in an artery upstream from a region within a heart associated with an MI. This location is ideal for releasing therapeutic factors such as those obtained by the method described herein, to enhance healing of the injured region.

Also provided is a system for delivering one or more biologic agents to a cardiovascular system including a heart and blood vessels, the heart having an injured myocardial region. The system includes a cardiovascular device including at least a portion configured for placement in the cardiovascular system, the cardiovascular device containing at least one biologic agent and configured to deliver the at least one biologic agent to the injured myocardial region. The at least one biologic agent is produced by the method described herein.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a biologic agent delivery system and portions of an environment in which the system is used.

FIG. 2 is an illustration of an embodiment of an in vitro biologic agent preparation system.

FIG. 3 is an illustration of an embodiment of a culturing apparatus of the in vitro biologic agent preparation system.

FIG. 4 is an illustration of an embodiment of a light source circuit of the culturing apparatus.

FIG. 5 is an illustration of an embodiment of a cardiovascular device allowing for delivery of a biologic agent.

FIG. 6 is an illustration of an embodiment of a transvascular device allowing for delivery of the biologic agent.

FIG. 7 is an illustration of an embodiment of another transvascular device allowing for delivery of the biologic agent.

FIG. 8 is an illustration of an embodiment of another transvascular device allowing for delivery of the biologic agent.

FIG. 9 is an illustration of an embodiment of another transvascular device allowing for delivery of the biologic agent.

FIG. 10 is an illustration of an embodiment of a stent allowing for delivery of the biologic agent.

FIG. 11 is an illustration of an embodiment of an epicardial patch allowing for delivery of the biologic agent.

FIG. 12 is a flow chart illustrating an embodiment of a method for preparing and delivering pro-growth or pro-survival factors to treat a cardiac injury.

FIG. 13 is a flow chart illustrating an embodiment of a method for preparing and delivering cells to treat the cardiac injury.

FIG. 14 is a graph of stem cell count under various conditions.

FIG. 15 shows data for stem cell counts under various conditions.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

### DEFINITIONS

As used herein, a light source which “substantially” emits a particular band or wavelength(s) of light is a light source in which more than 50%, preferably more than 60%, and more preferably more than 80%, of the band or wavelength(s) that are emitted are of the specified band or wavelength(s). For instance, a light source which emits substantially red light emits more than 50%, preferably more than 60%, and more preferably more than 80%, of the total light in the red portion of the spectrum.

The red portion of the spectrum is defined herein as wavelengths longer than about 600 nm, preferably longer than about 620 nm, and less than about 720 nm. A light that emits substantially infrared light is one in which more than 50%, preferably more than 60%, and more preferably more than 80%, of the total light in the infrared portion of the spectrum. The infrared portion of the spectrum is defined herein as light of wavelengths from about 720 nm to 1000 nm.

The terms “polypeptide,” “peptide,” and “protein” are used interchangeably to refer to polymers of amino acids of any length. These terms also include proteins that are post-translationally modified through reactions that include glycosylation, acetylation and phosphorylation.

By “growth factor” is meant an agent that, at least, promotes cell growth or induces phenotypic changes in cells.

“Biological agents” are those found in and/or expressed by cells, including proteins, glycoproteins, proteoglycans, and the like.

The term “isolated” when used in relation to a peptide or polypeptide or composition comprising a mixture of biological agents refers to a peptide or polypeptide or mixture that is separated from at least one contaminant nucleic acid, polypeptide or other biological component with which it is ordinarily associated in its natural source. For example, isolated peptide or polypeptide is present in a form or setting that is different from that in which it is found in nature, and an isolated composition of the invention is one which is free of intact cells.

The term “purified” or “to purify” means the result of any process that removes some of a contaminant from the component of interest, such as a protein or nucleic acid. The percent of a purified component is thereby increased in the sample.

As used herein, “pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a “substantially pure” composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

Methods and Systems

This document discusses methods to identify one or more wavelengths of light that induce or enhance production of pro-growth factors and/or pro-survival factors in mammalian cells, methods to prepare a composition that includes pro-growth factors and/or pro-survival factors, as well as method and system for treating injured tissue by using a composition including pro-growth factors and/or pro-survival factors, which composition is produced and isolated, and optionally purified, in vitro. In one embodiment, the present invention provides a method to enhance the production of desirable gene products. The method includes exposing cells in culture to selected bands or a wavelength of light for a period of time, so as to result in enhanced production of pro-growth factors and/or pro-survival factors. Thus, the method of the invention includes exposing a population of mammalian cells in vitro to one or more light sources that emit radiation at one or a plurality of wavelengths (i.e., in a particular spectral region or “band” which does not represent the spectrum emitted by white light) which stimulates (enhances or induces) expression or release from those cells factors which promote cell growth (pro-growth factors) and/or cell survival (pro-survival factors), e.g., radiation from 600 nm to 1000 nm (inclusive), including a specific wavelength or a specific band of wavelengths from 600 nm to 1000 nm. The exposure is for a period of time, e.g., 30 minutes to 1 hour or more, that results in increased amounts or levels of the pro-growth factors and/or pro-survival factors. Accordingly, the wavelength of light or band of light is selected as one which stimulates expression or release from cells of pro-growth factors and/or pro-survival factors. In one embodiment, red light, e.g., from 600 to 720 nm, is employed. In another embodiment, infrared light is employed, e.g., from 720 nm to 1000 nm. In yet another embodiment, both red and infrared light is employed.

In the methods of the present invention, one or more light sources may be employed. Thus, one light source may be used to emit one or a band of wavelengths. The method of the present invention can alternatively employ two, or more than two, light sources, each to emit the same of the different bands or wavelengths. The light source can comprise at least one incandescent lamp, a fluorescent lamp, a high sodium vapor lamp or a light emitting diode lamp, although the invention is not limited to those sources, as well as combinations of light sources, and, if necessary, a means to filter the spectrum of wavelengths which are emitted. In one embodiment, a single light source is employed to provide the desired band or wavelength of light. In one embodiment of the invention, one light source is employed that comprises an incandescent lamp with a red filter. In another embodiment, more than one light source is employed. In this embodiment, one light source may emit one band or wavelength of light and a second light source may emit a second band or wavelength of light.

Stem cells as well as other cells may be employed as a means of therapy to repair damaged organs such as the heart. For an example, after a heart attack, a portion of the heart is damaged and potentially dies as a result of a blocked artery. Thus, stem cells may help heal the damage caused by a myocardial infarction (MI).

To identify wavelengths of light useful to produce desirable factors, fibroblasts cells (mouse 3T3 cells) and swine bone marrow cells (swine 1844) were exposed to red light, e.g., 630 nm, green light, e.g., 525 nm, or blue light, e.g., 470 nm. Light wavelengths of 470 nm, 525 nm and 630 nm did not appear to enhance growth of swine bone marrow stem cells directly. In fact, blue light appeared to diminish stem cell growth at exposure times of 8 hours.

3T3 cells were disposed in Dulbecco's Modified Eagle's Medium, Modified formulation (Catalog No. 30-2002, American Type Culture Collection, Manassas, Va., U.S.A.), 10% calf serum, penicillin and streptomycin. The cells were placed in an incubator for 4 days and after changing the media, the cells were first placed in the dark for 2 days then exposed to a light source which was turned on for 1 hour. Then, 2 ml of media from the 3T3 cells were added to 5 ml of stem cell media containing swine bone marrow stem cells. The stem cell count was determined 5 days later. The procedure was repeated with (i) stem cell media only, (ii) stem cell media supplemented with media from 3T3 cells in an incubator for 6 days without light, (iii) stem cell media supplemented with media from 3T3 cells in an incubator for 4 days and then placed near a light source in the incubator for 2 days but without light exposure, (iv) stem cell media supplemented with media from 3T3 cells placed in an incubator for 4 days and then placed near a light source in the incubator for 2 days with 1 hour exposure to blue light, (v) stem cell media supplemented with media from 3T3 cells placed in an incubator for 4 days and then placed near a light source in the incubator for 2 days with 1 hour exposure to green light, and (vi) stem cell media supplemented with media from 3T3 cells placed in an incubator for 4 days and then placed in near a light source in the incubator for 2 days with 1 hour exposure to red light (1 hour at about 0.9 mW/cm² yields about 3.24 joules). The temperature of the media during light exposure increased linearly, e.g., from about 36° C. or about 37° C. to about 38° C. or about 40° C. over a 1 hour exposure.

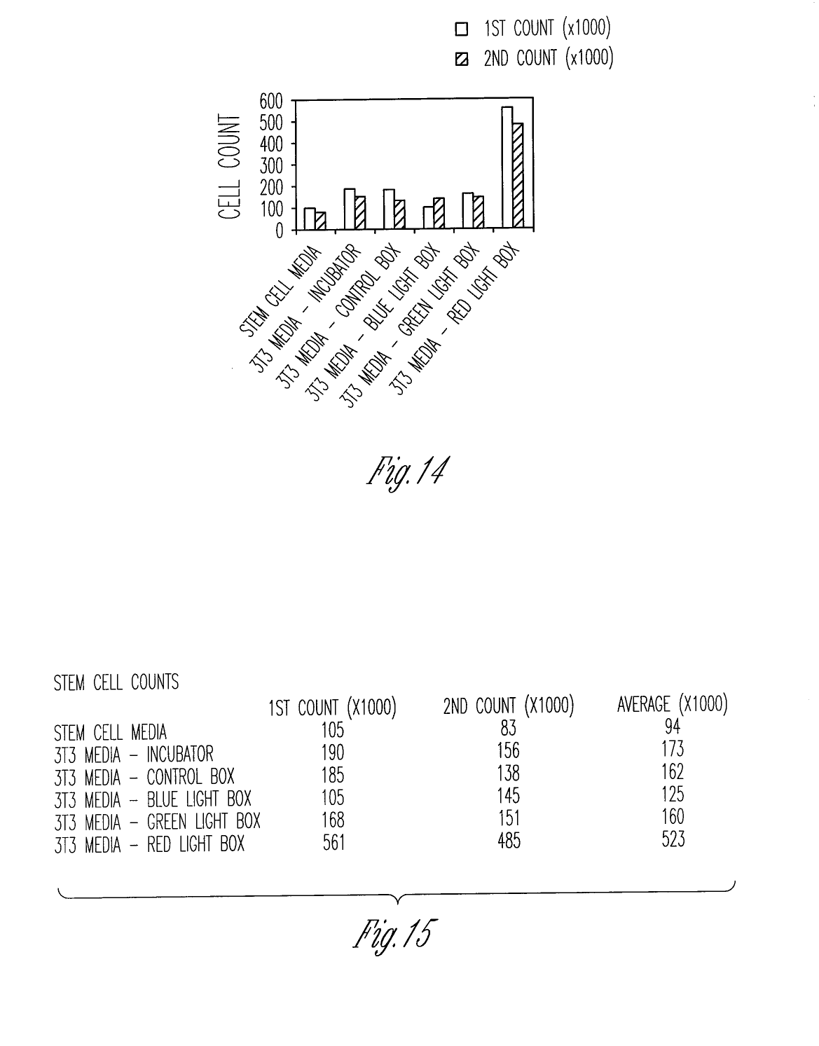

Swine cells supplemented with media from 3T3 cells exposed to red light had enhanced growth (2.5 to 3 times; FIGS. 14-15). Although exposing 3T3 cells to 1 hour of red light at about 0.9 mW/cm² resulted in expression of desirable factors, similar or improved results may be obtained using increased power and reduced exposure times. FIG. 14 is a graph showing an example of experimental results on growth response of swine bone marrow stem cells under various conditions, after receiving 2 ml of media from mouse fibroblast cells (3T3) that were first exposed to light for one hour. FIG. 15 shows the experimental results in stem cell counts. The experimental results shown in FIGS. 14 and 15 indicate that the supernatants from 3T3 cells exposed to red light contained pro-growth factors and/or pro-survival factors that enhanced stem cell growth. Thus, red light or infrared light alone, or a combination thereof may be employed to increase the amount or level of certain gene products in the extracellular environment of cells exposed to that light.

In one embodiment, the pro-growth factors and/or pro-survival factors in the composition of the invention are identified and once identified, prepared via recombinant means or chemical synthesis. Thus, a pro-survival factor or pro-growth factor which is a protein can be synthesized in vitro, e.g., by the solid phase peptide synthetic method or by recombinant DNA approaches. The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). These proteins, as well as proteins in the composition of the invention, can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, derivatives, e.g., chemically derived derivatives, of a given protein can be readily prepared. For example, amides of proteins may also be prepared by techniques well known in the art for converting a carboxylic acid group or precursor, to an amide. A preferred method for amide formation at the C-terminal carboxyl group is to cleave the protein from a solid support with an appropriate amine, or to cleave in the presence of an alcohol, yielding an ester, followed by aminolysis with the desired amine.

Salts of carboxyl groups of a protein may be prepared in the usual manner by contacting the protein with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of a protein may be prepared by utilizing an N-acyl protected amino acid for the final condensation, or by acylating a protected or unprotected protein. O-acyl derivatives may be prepared, for example, by acylation of a free hydroxy protein or protein resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the protein. Other amino-terminal modifications include aminooxypentane modifications (see Simmons et al., Science, 276, 276 (1997)).

In addition, the amino acid sequence of a protein can be modified so as to result in a variant. That is, one or more of the residues of the protein can be altered, so long as the resulting variant has substantially the same activity as that of the unmodified (functionally active) protein. For example, it is preferred that the variant has at least about 80% or more, at least 90%, the biological activity of the corresponding unmodified (native) protein, and at least about 80% or more, e.g., 85%, 90%, 95% or more, amino acid sequence identity to the corresponding native protein. Conservative amino acid substitutions are preferred. For example, aspartic-glutamic as acidic amino acids; lysine/arginine/histidine as basic amino acids; leucine/isoleucine/methionine/alanine/valine/glycine as hydrophobic amino acids; serine/threonine as hydrophilic amino acids. Conservative amino acid substitution also includes groupings based on side chains. In another example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant. Whether an amino acid change results in a functional protein can readily be determined by assaying the specific activity of the variant.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; trp, tyr, phe.

The invention also envisions variants with non-conservative substitutions. Non-conservative substitutions entail exchanging a member of one of the classes described above for another.

Acid addition salts of a protein or of amino residues of the protein may be prepared by contacting the protein or amine with one or more equivalents of the desired inorganic or organic acid, such as, for example, hydrochloric acid. Esters of carboxyl groups of the proteins may also be prepared by any of the usual methods known in the art.

The factors of the invention, whether or not identified or purified, may be employed in vitro or in vivo. In one embodiment, media containing the factors is collected from cells, e.g., fibroblasts, contacted with (exposed to) light, to yield a composition comprising one or more biologic agents including one or more pro-growth factors and/or one or more pro-survival factors. The composition may be purified, e.g., lyophilized, prior to use in vitro, e.g., the composition or a purified form thereof may be employed in vitro to treat cells to enhance the growth and/or survival and optionally the differentiation, of those cells, or in vivo, e.g., implanted into a region of injury within the body to promote cell growth of native (endogenous) cells, implanted (donor) cells, or recruited cells (either endogenous or donor cells), thereby enhancing healing of injured tissues.

In one embodiment, the composition or a purified form thereof is introduced to a mammal. In one embodiment, a dry (lyophilized) form may be injected or reconstituted prior to injection, into a MI or failing heart or other organ. For instance, a composition of the invention is injected to an injured region such as a MI, or to a vessel (artery or vein) upstream of injured tissue, or other injured regions, including injured organs, within a body. The amount administered is effective to enhance the growth and/or survival and optionally differentiation or implantation (engraftment) of endogenous cells or cells recruited to an injured region, or the growth and/or survival and optionally differentiation or engraftment of donor cells.

In one embodiment, a lyophilized composition may be reconstituted with an aqueous solvent, for instance, water to phosphate buffered saline.

In one embodiment, the composition or a purified form thereof is introduced to a biologic agent delivery device which is subsequently implanted into a mammal near or at an injured region. For instance, the composition is applied to a device such as a catheter, lead or stent, and may be released over a sustained time period, or may be administered during device placement.

In another embodiment, the composition is employed in vitro to treat donor cells, e.g., autologous donor cells, for cell therapy. The factor treated donor cells are introduced to an injured region, such as infarcted myocardium.

In one embodiment, the composition is administered in conjunction with cell therapy, e.g., to enhance growth and/or survival of donor cells. Sources for donor cells in cell-based therapies include skeletal muscle derived cells, for instance, skeletal muscle cells and skeletal myoblasts; cardiac derived cells, myocytes, e.g., ventricular myocytes, atrial myocytes, SA nodal myocytes, AV nodal myocytes, and Purkinje cells; bone marrow-derived cells, e.g., mesenchymal cells and stromal cells; smooth muscle cells; fibroblasts; SP cells; or pluripotent cells or totipotent cells, e.g., teratoma cells, hematopoietic stem cells, for instance, cells from cord blood and isolated CD34⁺ cells, multipotent adult progenitor cells, adult stem cells and embyronic stem cells. In one embodiment, the donor cells are autologous cells, however, non-autologous cells, e.g., xenogeneic cells, may be employed. The donor cells can be expanded in vitro to provide an expanded population of donor cells for administration to a recipient animal. In addition, donor cells may be treated in vitro as exemplified herein. Sources of donor cells and methods of culturing those cells are known to the art. See, for example, U.S. Pat. No. 5,130,141 and Jain et al. (Circulation, 103, 1920 (2001)), wherein the isolation and expansion of myoblasts from skeletal leg muscle is discussed (see also Suzuki et al., Circulation, 104, I-207 (2001), Douz et al., Circulation, III-210 (2000) and Zimmerman et al., Circulation Res., 90, 223 (2002)). Published U.S. Application 2002/0110910 discusses the isolation of and media for long term survival of cardiomyocytes. U.S. Pat. No. 5,580,779 discusses isolating myocardial cells from human atria and ventricles and inducing the proliferation of those myocardial cells. U.S. Pat. No. 5,103,821 discusses isolating and culturing SA node cells. For SA node cells, the cells may be co-cultured with stem cells or other undifferentiated cells. U.S. Pat. No. 5,543,318 discusses isolating and culturing human atrial myocytes. U.S. Pat. Nos. 6,090,622 and 6,245,566 discusses preparation of embryonic stem cells, while U.S. Pat. No. 5,486,359 discusses preparation of mesenchymal cells.

In one embodiment, biologic agent is applied to tissue by local administration. In one embodiment, the area including the damaged tissue is subjected to electrical and agent therapy, while in other embodiments the tissue is subjected to electrical therapy and cell therapy, e.g., by inserting or applying donor cells treated ex vivo with a composition of the invention.

Polymer Matrix

The polymer matrix may be formed of any physiologically compatible material which generally retains isolated protein(s) (which are charged molecules) or optionally other agents including other therapeutic agents under physiological conditions for a sustained period of time, e.g., for months or years, in the absence of an electrical field. The polymer matrix may extrude (release) isolated protein(s) in response to an electric field created by an electrical signal. The electric signal may be generated in response to the detection of a physiological signal associated with a condition, e.g., a cardiovascular condition.

The isolated protein(s) or optional other agent(s) may be introduced to a solution of monomers prior to polymerization or to the polymer matrix, e.g., dissolved in a solvent (e.g., water, propylene, glycol, etc.) and the resulting solution can be incorporated into the polymer matrix material. Once the isolated protein(s) are embedded in or applied to a polymer matrix, the resulting mixture may be introduced to an implantable device. Alternatively, the polymer matrix may be first coupled to an implantable device and then the isolated protein(s) embedded in or applied thereto, either passively or actively (through, for example, such methods as iontophoresis). Upon delivery of an electric field, the isolated protein(s) or optional other agent(s) are released from the matrix at a rate which is greater than the rate of release in the absence of the electric field. In particular, the isolated protein(s) are released to adjacent cells or tissue or the vessel lumen in response to an electric field generated by a device, which release is in an amount proportional to the applied electric field. Once the electric signal is stopped, the proteins are no longer released or released at a rate which is significantly reduced relative to the rate of release in the presence of the electric field.

The matrix materials will preferably be physiologically inert and capable of retaining the charged molecule to be delivered. Matrix materials which may be used include: polyacetic or polyglycolic acid and derivatives thereof, polyorthoesters, polyesters, polyurethanes, polyamino acids such as polylysine, lactic/glycolic acid copolymers, polyanhydrides and ion exchange resins such as sulfonated polytetrafluorethylene, or combinations thereof.

Biocompatible materials useful in the invention include those that are substantially nonbiodegradable, i.e., those for retention of at least one component of their contents, and those that are biodegradable, i.e., those for sustained release of at least one component of their contents. The biocompatible material may contain, or be embedded and/or coated, with donor cells or isolated proteins, and may be suitable for retaining and/or immobilizing donor cells or optionally other agents including other therapeutic agents under physiological conditions for a sustained period of time, e.g., for months or years after implantation.

The biocompatible material may be a semipermeable membrane which allows the transport of isolated proteins and prevents entry and exit of large molecules, e.g., entry of undesirable molecules such as antibodies and immune cells, and exit of the donor cells. Molecular weight cut offs for the semipermeable membrane may be about 50 to 100 kD. The membrane may be made of any suitable material which is nondegradable and biocompatible, e.g., agarose, polyvinyl alcohol, e.g., cross-linked polyvinyl alcohol, polyacrylates, polyamides, and polyurethane, and including a dialysis membrane, nylon or cellulose, e.g., cellulose acetate or methyl cellulose, including those which are derivatized to decrease degradation in vivo. The semipermeable materials may also be conjugated with heparin and/or polyethylene glycol (PEG) to decrease immunogenic response, blood clotting and cell attachment on the surface. Examples of such enclosures and semipermeable membranes are discussed in U.S. Pat. No. 5,593,852; U.S. Pat. No. 5,431,160: U.S. Pat. No. 5,372,133; U.S. Pat. No. 4,919,141, and U.S. Pat. No. 4,703,756.

Biocompatible materials may include polyglycolic acid and derivatives thereof, carboxyl containing polymers, polyorthoesters, polyesters, e.g., hydroxyl containing polyesters, polyurethanes, polyamino acids such as polylysine, lactic/glycolic acid copolymers, polyanhydrides and ion exchange resins such as sulfonated polytetrafluorethylene, polydimethyl siloxanes (silicone rubber) or combinations thereof. Reactive groups on the polymers, for example, hydroxy or amino groups, can be acetylated (e.g., polymer-O—C(═O)CH₃ or polymer-NR—C(═O)CH₃), and those groups can be prepared either before or after polymerization of monomers.

Additionally, the biocompatible material may be formed from natural proteins or materials which optionally may be modified, e.g., crosslinked using a crosslinking agent such as 1-ethyl-3-(3-dimethylamino-propyl)carbodiimide hydrochloride. Such natural materials include polysaccharides, e.g., cellulose including regenerated cellulose (rayon), albumin, collagen, fibrin, alginate, extracellular matrix (ECM), e.g., xenogeneic ECM, hyaluronan, chitosan, gelatin, keratin, potato starch hydrolyzed for use in electrophoresis, and agar-agar (agarose), or other “isolated materials”. An “isolated” material has been separated from at least one contaminant structure with which it is normally associated in its natural state such as in an organism or in an in vitro cultured cell population.

In one embodiment, the material may include liposomes, a hydrogel, cyclodextrins, nanocapsules or micro spheres. Thus, a biocompatible material includes synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[α(4-aminobutyl)]-1-glycolic acid, polyethylene oxide (Roy et al., Mol. Ther., 7:401 (2003)), poly orthoesters (Heller et al., Adv. Drug Delivery Rev., 54:1015 (2002)), silk-elastin-like polymers (Megeld et al., Pharma. Res., 19:954 (2002)), alginate (Wee et al., Adv. Drug Deliv. Rev., 31:267 (1998)), EVAc (poly(ethylene-co-vinyl acetate), microspheres such as poly(D, L-lactide-co-glycolide) copolymer and poly(L-lactide) and poly glycolide, polydioxenone, poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, an agarose hydrogel, or a lipid microtubule-hydrogel.

In one embodiment, the isolated protein or donor cells are encapsulated by, embedded in or applied to a biocompatible material, e.g., a nonbiodegradable or biodegradable material, respectively, including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols.

In some embodiments, isolated protein or the donor cells are embedded in or encapsulated by a biocompatible and biodegradable polymeric such as collagen, fibrin, polyhydroxyalkanoates, cellulose, polylactic-polyglycolic acid, or a polyanhydride.

Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), poly(dioxanone) (PPS) or cellulose derivatives such as cellulose acetate. In an alternative embodiment, a biologically derived polymer, such as protein, collagen, e.g., hydroxylated collagen, or fibrin, or polylactic-polyglycolic acid or a polyanhydride, is a suitable polymeric matrix material.

In another embodiment, the biocompatible material includes polyethyleneterephalate, polytetrafluoroethylene, copolymer of polyethylene oxide and polypropylene oxide, a combination of polyglycolic acid and polyhydroxyalkanoate, or gelatin, alginate, collagen, hydrogels, poly-3-hydroxybutyrate, poly-4-hydroxybutyrate, and polyhydroxyoctanoate, and polyacrylonitrilepolyvinylchlorides.

The following polymers may be employed with donor cells, e.g., natural polymers such as starch, chitin, glycosaminoglycans, e.g., hyaluronic acid, dermatan sulfate and chrondrotin sulfate, collagen, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone. The incorporation of molecules such as tricalciumphosphate, hydroxyapetite and basic salts into a polymer matrix can alter the degradation and resorption kinetics of the matrix. Moreover, the properties of polymers can be modified using cross-linking agents.

In one embodiment, the biocompatible material is isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like. The preparation and use of isolated ECM in vivo is described in co-pending, commonly assigned U.S. patent application Ser. No. 11/017,237, entitled “USE OF EXTRACELLULAR MATRIX AND ELECTRICAL THERAPY,” filed on Dec. 20, 2004, which is hereby incorporated by reference in its entirety.

In another embodiment, the biocompatible material is a synthetic, nonbiodegradable polymer such as polyurethanes, polydimethylsiloxanes (silicone rubbers), ethylene vinyl acetate copolymer (EVA), poly methylmethacrylate, polyamides, polycarbonates, polyesters, polyethylene, polypropylenes, polystyrenes, polyvinyl chloride, polyvinyl alcohols, polytetrafluoroethylene, or cellulose derivatives such as cellulose acetate.

In one embodiment, the biocompatible material acts as a selectively semipermeable membrane such as a dialysis membrane or nylon.

Compositions, Dosages and Routes of Administration

The amount of agent administered will vary depending on various factors including, but not limited to, the agent chosen, the disease, whether prevention or treatment is to be achieved, and if the agent is modified for bioavailability and in vivo stability. Thus, the agents of the invention may be employed in conjunction with other therapies, e.g., therapies for ischemia, including gene therapies and/or cell therapies, e.g., see U.S. patent application Ser. No. 10/723,258, filed on Nov. 25, 2003, entitled “METHOD AND APPARATUS FOR CELL AND ELECTRICAL THERAPY OF LIVING CELLS” and U.S. patent application Ser. No. 10/788,906, filed on Feb. 27, 2004, entitled “METHOD AND APPARATUS FOR DEVICE CONTROLLED GENE EXPRESSION”, the disclosures of which are incorporated herein by reference in their entirety.

Administration of the agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms comprising the agents of the invention, which, as discussed below, may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes, although local administration of at least one agent, e.g., via an implantable device, is a preferred embodiment of the invention. The formulations may, where appropriate, be conveniently presented in discrete unit dosage forms and may be prepared by any of the methods well known to pharmacy. Such methods may include the step of bringing into association the agent with liquid carriers, solid matrices, semi-solid carriers, finely divided solid carriers or combinations thereof, and then, if necessary, introducing or shaping the product into the desired delivery system.

Pharmaceutical formulations containing the agents of the invention can be prepared by procedures known in the art using well known and readily available ingredients. For example, the agent can be formulated with common excipients, diluents, or carriers. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols. The formulations can include buffering agents such as calcium carbonate, magnesium oxide and magnesium carbonate, as well as, inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and gelatin, microcrystalline cellulose, sodium lauryl sulfate, starch, talc, or titanium dioxide, or liquid vehicles such as polyethylene glycols (PEGs) and vegetable oil.

The pharmaceutical formulations of the agents of the invention can also take the form of an aqueous or anhydrous solution or dispersion, or alternatively the form of an emulsion or suspension.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

Additionally, the agents are well suited to formulation as sustained release dosage forms and the like. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, epicardial patch, leads, and the like.

The formulations and compositions described herein may also contain other ingredients such as antimicrobial agents, or preservatives. Furthermore, as described herein the active ingredients may also be used in combination with other therapeutic agents, or therapies, for instance, cell therapy.

The number of cells to be administered will be an amount which results in a beneficial effect to the recipient. For example, from 10² to 10¹⁰, e.g., from 10³ to 10⁹, 10⁴ to 10⁸, or 10⁵ to 10⁷, cells can be administered to, e.g., injected, the region of interest, for instance, infarcted and tissue surrounding infarcted tissue. The amount of pro-growth factor and/or pro-survival factor to be administered may be, for example, from about 1 ng to about 10 mg, e.g., from about 1 μg to about 1 mg. Other agents which may enhance cardiac function or have other beneficial effects may optionally be present in a composition comprising the pro-growth factors and/or pro-survival factors or in a composition with donor cells, or administered separately.

The isolated pro-growth factors and/or pro-survival factors or donor cells may be administered during a prophylactic, diagnostic or therapeutic vascular procedure or an invasive or minimally invasive surgical procedure.

While treatment of myocardial tissue injured by ischemia or infarction is discussed as a specific example, the present subject matter is generally applicable in promoting healing of injured cardiac and non-cardiac tissues.

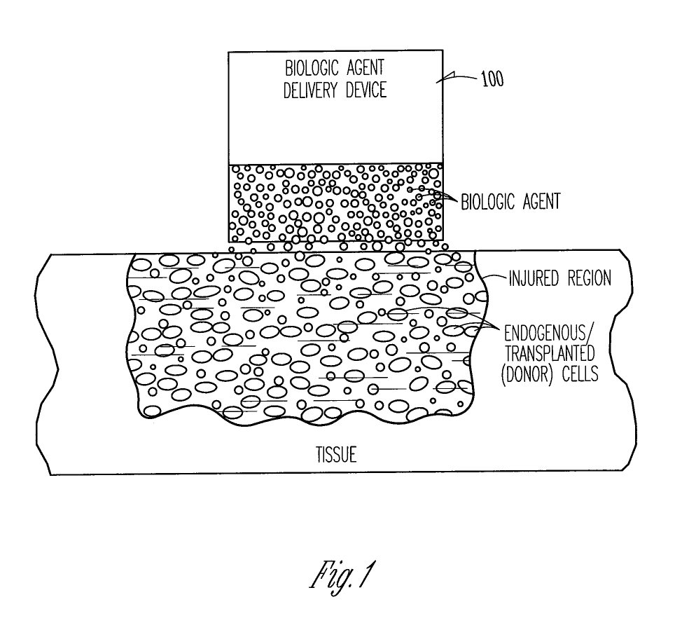

FIG. 1 is an illustration of an embodiment of a biologic agent delivery system 100 and portions of an environment in which system 100 is used. System 100 includes a biologic agent delivery device that contains one or more biologic agents and delivers the one or more biologic agents to an injured region to promote tissue healing. The one or more biologic agents include pro-growth factors and/or pro-survival factors produced in vitro by exposing cells, such as fibroblast cells in growth media, to light. The light has one or more wavelengths predetermined for inducing the cells to express the pro-growth factors and/or pro-survival factors. The one or more biologic agents promote survival and growth, and optionally differentiation or engraftment, of endogenous and/or transplanted (donor) cells in the injured region.

FIG. 1 is a general conceptual illustration of the present subject matter and does not represent any specific shape or structure of the biologic agent delivery device and any particular type of tissue. In various embodiments, the shape and structure of the biologic agent delivery device depend on anatomic constraints and delivery strategies. In one embodiment, which is specifically discussed in this document by way of example, but not by way of limitation, the biologic agent delivery device is a cardiovascular device, which is a device including at least a portion configured for placement in the cardiovascular system (including the heart and blood vessels). The one or more biologic agents are delivered to an injured myocardial region to promote myocardial tissue healing after an ischemic event such as an acute myocardial infarction. The cardiovascular device delivers the one or more biologic agents directly to the injured myocardial region or to a location in the cardiovascular system upstream from the injured myocardial region.

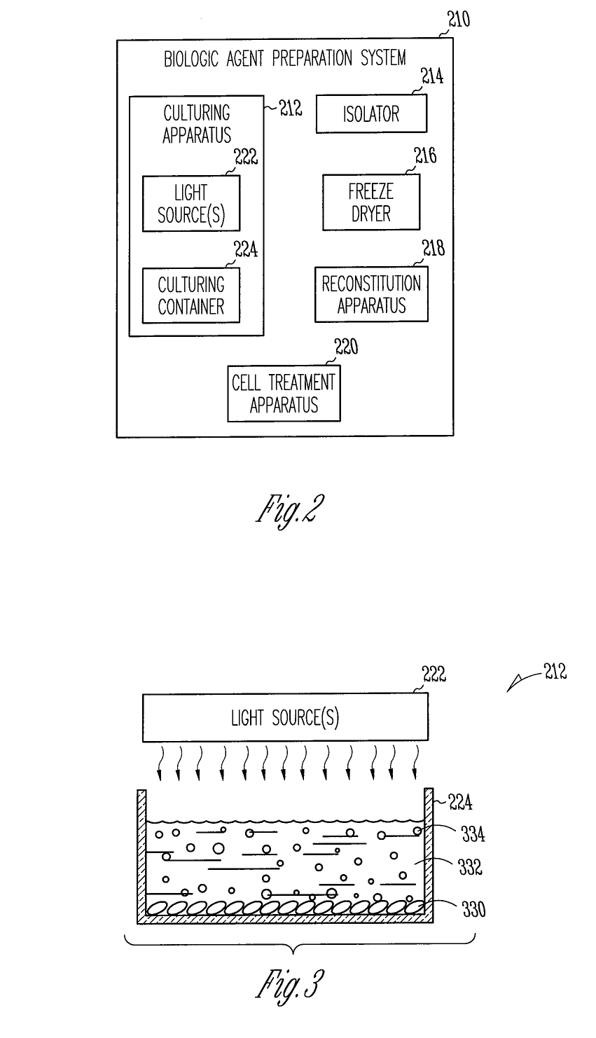

FIG. 2 is an illustration of an embodiment of a biologic agent preparation system 210 for in vitro preparation of a biologic agent used in a cardiac biologic therapy. System 210 includes a culturing apparatus 212, an isolator 214, a freeze dryer 216, a reconstitution apparatus 218, and a cell treatment apparatus 220. In various embodiments, depending on the therapy strategy and the required or desirable form of the biologic agent, system 210 includes culturing apparatus 212, an isolator 214 and optionally one or more of freeze dryer 216, reconstitution apparatus 218, and cell treatment apparatus 220.

Culturing apparatus 212 includes one or more light sources 222 and a culturing container 224. Culturing container 224 includes a first population of cells and growth media. In one embodiment, the first population of cells includes fibroblast cells. In a specific embodiment, the first population of cells includes autologous fibroblast cells. In another specific embodiment, the first population of cells includes nonautologous fibroblast cells, e.g., cells from another person or a different species. Light source(s) 222 induce the first population of cells to produce pro-growth factors and/or pro-survival factors, which factors are released from the cells, e.g., into the media. The pro-growth factors and/or pro-survival factors enhance survival and/or growth in cells, e.g., cells that may be present in a mammal (endogenous cells) or cells transplanted into (donor cells) an injured myocardial region of a mammal. Examples of the second type of cells include stem cells, cardiac cells, and endothelial cells. The enhanced survival and/or growth, and optionally differentiation or engraftment, of endogenous and/or transplanted cells provides for repair of the injured myocardial region. In one embodiment, light source(s) 222 include one or more light sources each emitting a light having a wavelength of approximately 400 nanometers (nm) and 1000 nm and an intensity between approximately 1000 millicandela (mcd) and 10,000 mcd, or in combination, approximately 0.1 to about 50 mW/cm². In one embodiment, cells are exposed to a light source(s) for a time to deliver from about 0.1 to about 10 joules, with 3 joules being a specific example. In one embodiment, light source(s) 222 include a plurality of light sources having substantially different optical wavelengths. In a specific embodiment, light source(s) 222 include at least a red light source emitting a red light having a wavelength of approximately 600 nm to 720 nm, with approximately 630 nm being a specific example. In another specific embodiment, light source(s) 222 include at least an infrared light source emitting an infrared light having a wavelength of approximately 720 nm to 1000 nm, with approximately 880 nm being a specific example. In another specific embodiment, light source(s) 222 include a plurality of light sources including at least the red light source and the infrared light source.

Isolator 214 allows the pro-growth factors and/or pro-survival factors to be isolated by separating the first population of cells from the pro-growth factors and/or pro-survival factors, e.g., by separating the first population of cells from media. In one embodiment, isolator 214 includes a filtering apparatus. After the pro-growth factors and/or pro-survival factors are produced and released into the media, the content of culturing container 224 is filtered to remove the first population of cells, and, if needed, filtered again to increase the concentration of the factors. In one embodiment, after the steps of filtering, the pro-growth factors and/or pro-survival factors are further purified, for instance, by methods well-known to the art including but not limited to fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography, and the like.

To provide a dry form of the isolated pro-growth factors and/or pro-survival factors, freeze dryer 216 may be employed to lyophilize the isolated pro-growth factors and/or pro-survival factors. The lyophilized pro-growth factors and/or pro-survival factors are in the form of dry powder. In one embodiment, the lyophilized pro-growth factors and/or pro-survival factors are embedded into another material that is coated or otherwise incorporated into a device or a portion of a device that is placed in the injured myocardial region or a cardiovascular location upstream from the injured myocardial region.

To provide a liquid form of the pro-growth factors and/or pro-survival factors from the dry form, reconstitution apparatus 218 is used to reconstitute the lyophilized pro-growth factors and/or pro-survival factors. In one embodiment, the reconstituted pro-growth factors and/or pro-survival factors are injected into the injured myocardial region or a cardiovascular location upstream from the injured myocardial region.

When donor cells are to be delivered to an injured myocardial region, instead of or in addition to delivering the pro-survival and/or pro-growth factors to the injured myocardial region, cell treatment apparatus 220 which contains a culturing container with those cells may be employed. Examples of donor cells include, but are not limited to, stem cells, cardiac cells, and endothelial cells. Thus, prior to administration into tissue, donor cells are treated with the pro-growth factors and/or pro-survival factors in cell treatment apparatus 220. The treated cells have an enhanced ability to survive and grow, and optionally differentiate or engraft, after being transplanted into the injured myocardial region. An example of method and apparatus for administration of cells into cardiac tissue is discussed in U.S. patent application Ser. No. 10/722,115, “METHOD AND APPARATUS FOR CELL AND ELECTRICAL THERAPY OF LIVING TISSUE,” filed on Nov. 25, 2003, assigned to Cardiac Pacemakers, Inc., which is incorporated herein by reference in its entirety.

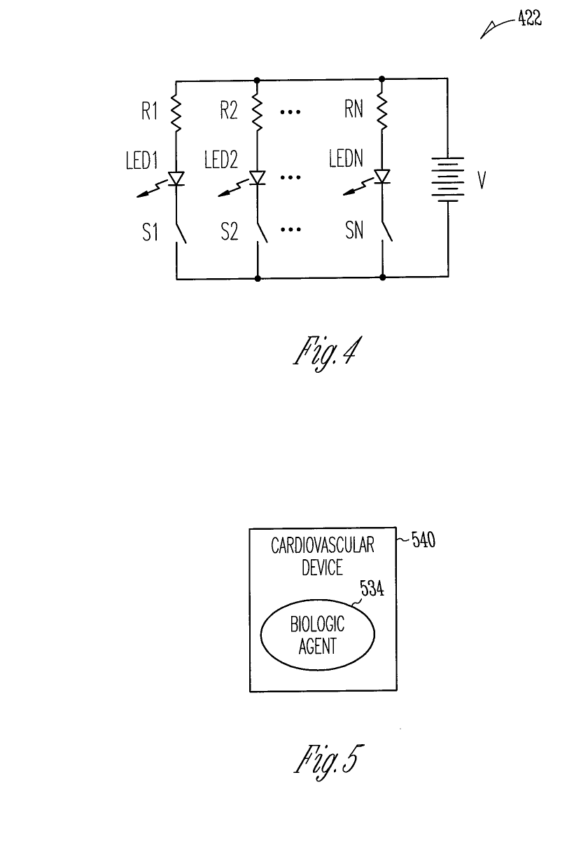

FIG. 3 is an illustration of an embodiment of culturing apparatus 212, including light source(s) 222 and culturing container 224. First population of cells 330, such as fibroblast cells, is disposed into growth media 332 contained in culturing container 224. In response to the light emitting from light source(s) 222, cells 330 produce pro-growth factors and/or pro-survival factors 334, which are released into growth media 332.

FIG. 4 is an illustration of an embodiment of a light source circuit 422, which is a specific embodiment of the circuit of light source(s) 222. Light source circuit 422 includes one or more light-emitting diodes (LEDs) each functioning as a light source. In one embodiment, each of the one or more LEDs emits a light having one or more specified wavelengths or ranges of wavelength. In another embodiment, each of the one or more LEDs is provided with an optical filter to produce the light having a specified wavelength or range of wavelength.

In one embodiment, as illustrated in FIG. 4, light source circuit 422 includes a plurality of LEDs, LED1 through LEDN, connected in parallel and powered by a voltage source V. Each of the LEDs is current-limited using a resistor R and is turned on or off using a switch S. In one embodiment, LED1 through LEDN are of the same type and emit a light with a single wavelength, such as a red light or an infrared light. In another embodiment, LED1 through LEDN include two or more types of LEDs emitting lights having substantially different wavelengths. This allows selection of one or more wavelengths for inducing the production of the pro-growth factors and/or pro-survival factors depending on particular type of cells response to particular types of light.

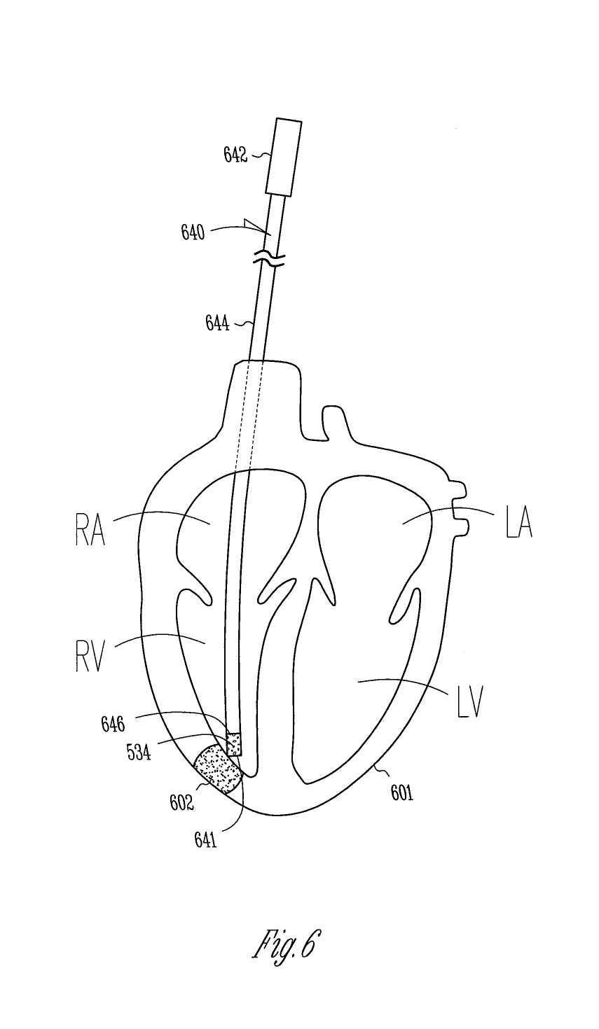

FIG. 5 is an illustration of an embodiment of a cardiovascular device 540 that contains a biologic agent 534. Cardiovascular device 540 is capable of delivering one or more biologic agents to a cardiovascular system, including the heart and blood vessels. The one or more biologic agents are delivered to treat an injured myocardial region, which may result from an ischemic event such as acute myocardial infarction. Cardiovascular device 540 includes at least a portion configured for placement in the cardiovascular system to deliver biologic agent 534 to the injured myocardial region. Biologic agent 534 including the pro-growth factors and/or pro-survival factors produced in vitro by exposing cells in growth media to a light that induces the cells to express the pro-growth factors and/or pro-survival factors, as discussed above.

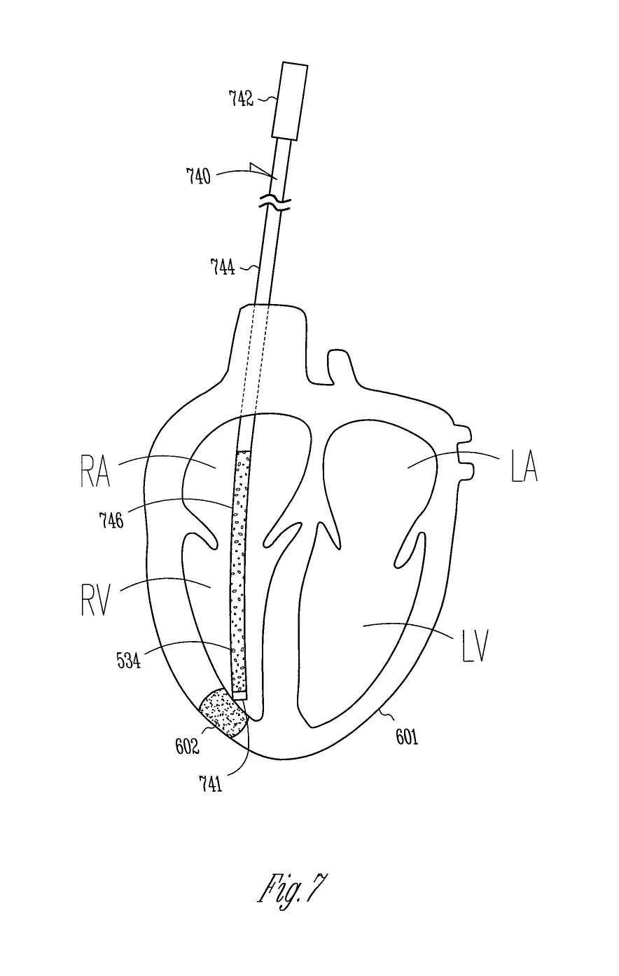

FIGS. 6-11 illustrate, by way of example, but not by way of limitation, various embodiments of cardiovascular device 540. In various embodiments, cardiovascular device 540 is capable of delivering biologic agent 534 directly to the injured myocardial region or to one or more locations in the cardiovascular system upstream from the injured myocardial region. In various embodiments, cardiovascular device 540 allows for injection of biologic agent 534. In various other embodiments, cardiovascular device 540 allows for gradual release of biologic agent 534. The pro-growth factors and/or pro-survival factors of biologic agent 534 promote healing of the injured myocardial region.

FIG. 6 is an illustration of an embodiment of a transvascular device 640, which is a specific embodiment of cardiovascular device 540. Transvascular device 640 includes a distal end 641, a proximal end 642, and an elongate body 644 between distal end 641 and proximal end 642. Distal end 641 is configured to reach a cardiac location accessible through a blood vessel. In one embodiment, transvascular device 640 is an implantable cardiac stimulation lead including one or more electrodes at or near distal end 641 for delivering pacing and/or cardioversion/defibrillation pulses. The one or more electrodes are each connected to a conductor extending through elongate body 644 and connected to a lead connector at proximal end 642. The lead connector is configured to connect to an implantable cardiac rhythm management device. In another embodiment, transvascular device 640 is a percutaneous transvascular catheter.

Transvascular device 640 includes an agent delivery collar 646 at distal end 641. As illustrated in FIG. 6, distal end 641 is placed upon an injured myocardial region 602 in a heart 601. Agent delivery collar 646 contains biologic agent 534 and releases biologic agent 534 to injured myocardial region 602. A specific example of an implantable pacing lead including an agent delivery collar is discussed in U.S. patent application Ser. No. 10/745,302, “HIS BUNDLE MAPPING, PACING, AND INJECTION METHOD AND LEAD,” filed on Dec. 23, 2003, assigned to Cardiac Pacemakers, Inc., which is incorporated herein by reference in its entirety. In one embodiment, in addition to the pro-growth factors and/or pro-survival factors, cardiac pacing promotes the survival, growth and optionally differentiation or engraftment of cardiac cells, such as in a way discussed in U.S. patent application Ser. No. 10/722,115. In another embodiment, cardiac pacing pulses are delivered to injured myocardial region 602 using transvascular device 640, which is a pacing lead, in a cardiac remodeling control therapy that pre-excites a portion of heart 601 including injured myocardial region 602 after a myocardial infarction.

In one embodiment, agent delivery collar 646 is made of a polymeric material in which biologic agent 534 is embedded. In a specific embodiment, biologic agent 534 includes the lyophilized pro-growth factors and/or pro-survival factors. In one embodiment, agent delivery collar 646 allows for controllable release of biologic agent 534. In a specific embodiment, the polymeric material is an electrically sensitive polymer having a structure being electrically controllable by applying an electric field. In one embodiment, the porosity of the electrically sensitive polymer is a function of an electric field applied on the electrically sensitive polymer. In other words, the electrically sensitive polymer includes pores with sizes being a function of the electric field. The electrically sensitive polymer enters a substantially porous state upon application of an electrical field of certain amplitude and enters a substantially non-porous state upon removal of that electric field or a change of its amplitude. In another embodiment, the binding affinity of the electrically sensitive polymer is a function of the electric field. In other words, the degree to which the factors are attracted to the electrically sensitive polymer is a function of the electric field. The electrically sensitive polymer enters a state of low binding affinity upon application of an electrical field of certain amplitude and enters a state of high binding affinity upon removal of that electric field or a change of its amplitude. In both embodiments, the release of biologic agent 534 is controllable by controlling the amplitude of an electrical field, such as using the one or more electrodes at or near distal end 641.

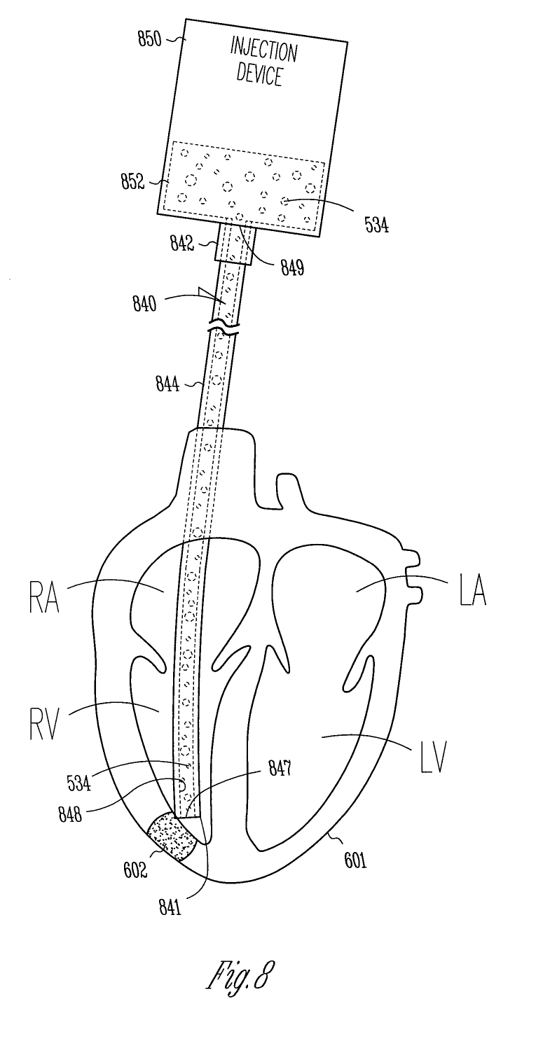

FIG. 7 is an illustration of an embodiment of another transvascular device 740, which is another specific embodiment of cardiovascular device 540. Transvascular device 740 includes a distal end 741, a proximal end 742, and an elongate body 744 between distal end 741 and proximal end 742. Distal end 741 is configured to reach a cardiac location accessible through a blood vessel. In one embodiment, transvascular device 740 is an implantable cardiac stimulation lead including one or more electrodes at or near distal end 741 for delivering pacing and/or cardioversion/defibrillation pulses. The one or more electrodes are each connected to a conductor extending through elongate body 744 and connected to a lead connector at proximal end 742. The lead connector is configured to connect to an implantable cardiac rhythm management device. In another embodiment, transvascular device 740 is a percutaneous transvascular catheter.

Transvascular device 740 includes a coating 746 that covers portions of distal end 741 and/or elongate body 744. As illustrated in FIG. 7, distal end 741 is placed upon injured myocardial region 602 in heart 601. Coating 746 contains biologic agent 534 and releases biologic agent 534 to injured myocardial region 602 as well as endocardial spaces upstream from injured myocardial region 602.

In one embodiment, coating 746 is made of a polymeric material in which biologic agent 534 is embedded. The polymeric material is suitable for coating a transvascular device. In a specific embodiment, biologic agent 534 includes the lyophilized pro-survival or pro-growth factors. In one embodiment, coating 746 allows for controllable release of biologic agent 534. In a specific embodiment, the polymeric material is the electrically sensitive polymer discussed above with reference to FIG. 6. The release of biologic agent 534 is controllable by controlling the amplitude of an electrical field, such as using the one or more electrodes at or near distal end 741 or one or more conductors extending through elongate body 744.

FIG. 8 is an illustration of an embodiment of another transvascular device 840, which is another specific embodiment of cardiovascular device 540. Transvascular device 840 includes a distal end 841, a proximal end 842, and an elongate body 844 between distal end 841 and proximal end 842. Distal end 841 is configured to reach a cardiac location accessible through a blood vessel. In one embodiment, transvascular device 840 is an implantable cardiac stimulation lead including one or more electrodes at or near distal end 841 for delivering pacing and/or cardioversion/defibrillation pulses. The one or more electrodes are each connected to a conductor extending through elongate body 844 and connected to a lead connector at proximal end 842. The lead connector is configured to connect to an implantable cardiac rhythm management device. In another embodiment, transvascular device 840 is a percutaneous transvascular catheter.

Transvascular device 840 includes a lumen 848 that extends within elongate body 844 from proximal end 842 to distal end 841. Lumen 848 has a distal opening 847 at distal end 841 and a proximal opening 849 at proximal end 842. An injection device 850 includes a chamber 852 containing biologic agent 534. Proximal opening 849 is connected to injection device 850 such that when being injected, biologic agent 534 enters lumen 848 through proximal opening 849, flows through lumen 848, and exits through distal opening 847 to enter injured myocardial region 602. Examples of injection device 850 include an agent pump within the implantable cardiac rhythm management device and an external injection device such as a syringe. The external device is used during a catheterization procedure, with transvascular device 840 being a percutaneous transvascular catheter, or during an implantation procedure, with transvascular device 840 being an implantable cardiac stimulation lead. A specific example of an implantable pacing lead having a lumen allowing for delivery of an agent is discussed in U.S. patent application Ser. No. 10/745,302.

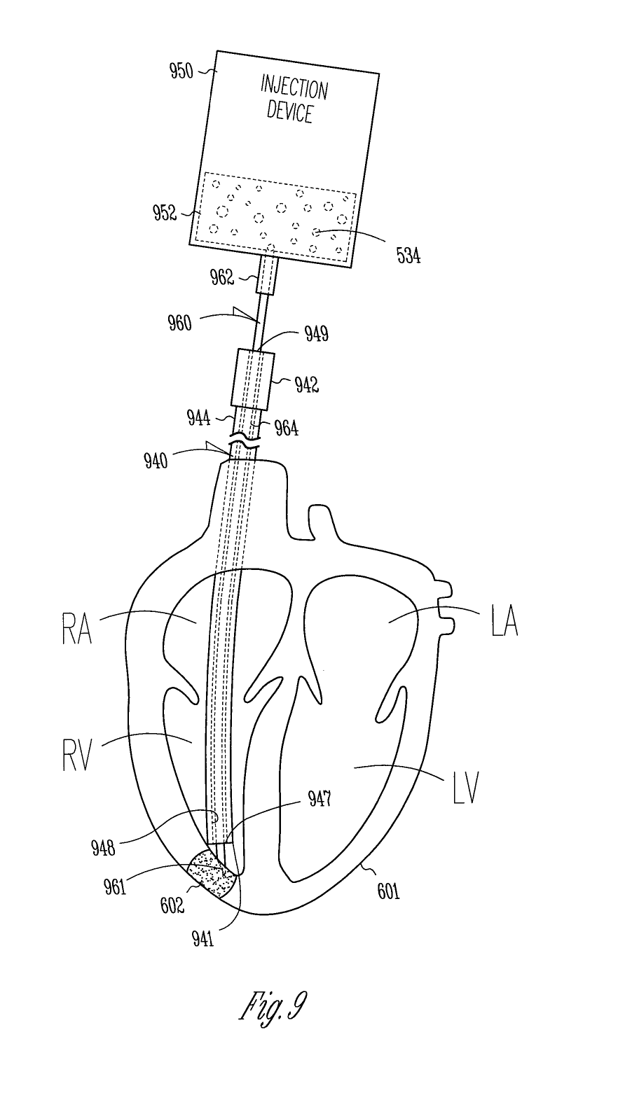

FIG. 9 is an illustration of an embodiment of another transvascular device 940, which is another specific embodiment of cardiovascular device 540. Transvascular device 940 includes a distal end 941, a proximal end 942, and an elongate body 944 between distal end 941 and proximal end 942. Distal end 941 is configured to reach a cardiac location accessible through a blood vessel. In one embodiment, transvascular device 940 is an implantable cardiac stimulation lead including one or more electrodes at or near distal end 941 for delivering pacing and/or cardioversion/defibrillation pulses. The one or more electrodes are each connected to a conductor extending through elongate body 944 and connected to a lead connector at proximal end 942. The lead connector is configured to connect to an implantable cardiac rhythm management device. In another embodiment, transvascular device 940 is a percutaneous transvascular catheter.

Transvascular device 940 includes a lumen 948 that extends within elongate body 944 from proximal end 942 to distal end 941. Lumen 948 has a distal opening 947 at distal end 941 and a proximal opening 949 at proximal end 942. Lumen 948 is configured to accommodate at least a portion of a flexible hollow needle 960, which is used for injection of biologic agent 534 from an external injection device 950. Flexible hollow needle 960 has a needle tip 961, a rear end 962, and a flexible needle body 964. To inject biologic agent 534, needle tip 961 enters proximal opening 949 and passes through lumen 948 to exit from distal opening 947 to enter injured myocardial region 602. Using flexible hollow needle 960 allows for injection of biologic agent 534 into the cardiac wall of heart 601. Injection device 950 includes a chamber 952 that contains biologic agent 534. Examples of injection device 950 include an external agent pump and a syringe. The external device is used during a catheterization procedure, with transvascular device 940 being a percutaneous transvascular catheter, or during an implantation procedure, with transvascular device 940 being an implantable cardiac stimulation lead. A specific example of an implantable pacing lead having a lumen accommodating portions of a flexible hollow needle is discussed in U.S. patent application Ser. No. 10/745,302.

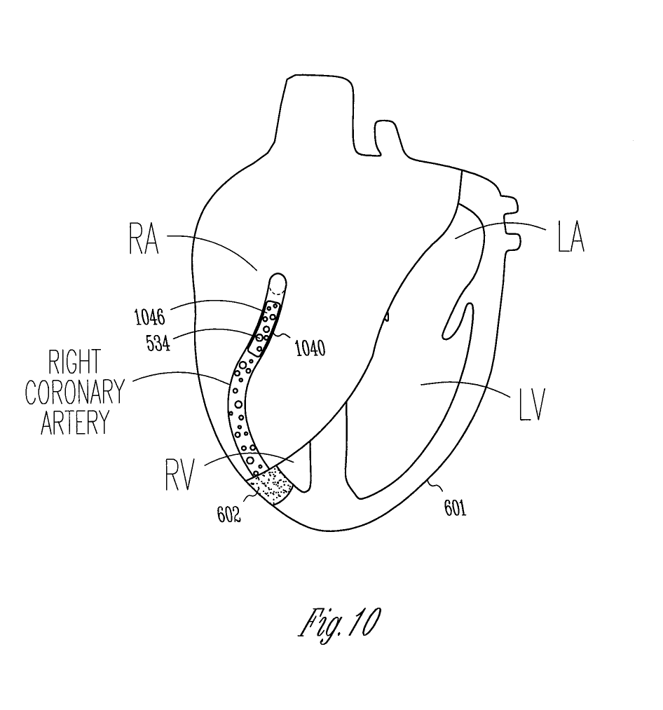

FIG. 10 is an illustration of an embodiment of a stent 1040, which is another specific embodiment of cardiovascular device 540. Stent 1040 includes at least a portion covered by a coating 1046, in which biologic agent 534 is embedded.

In one embodiment, coating 1046 is made of a polymeric material in which biologic agent 534 is embedded. In a specific embodiment, biologic agent 534 includes the lyophilized pro-survival or pro-growth factors. In one embodiment, coating 1046 allows for controllable release of biologic agent 534. In a specific embodiment, the polymeric material is the electrically sensitive polymer discussed above with reference to FIG. 6. The release of biologic agent 534 is controllable by controlling the amplitude of an electrical field, such as using pacing electrodes, if available.

Stent 1040 is used to deliver biologic agent 534 if it is to be placed in a vascular location upstream from the myocardial region to be treated. In one example, as illustrated in FIG. 10, stent 1040 is placed in the right coronary artery in a location upstream from injured myocardial region 602, which is near the right ventricular apex.

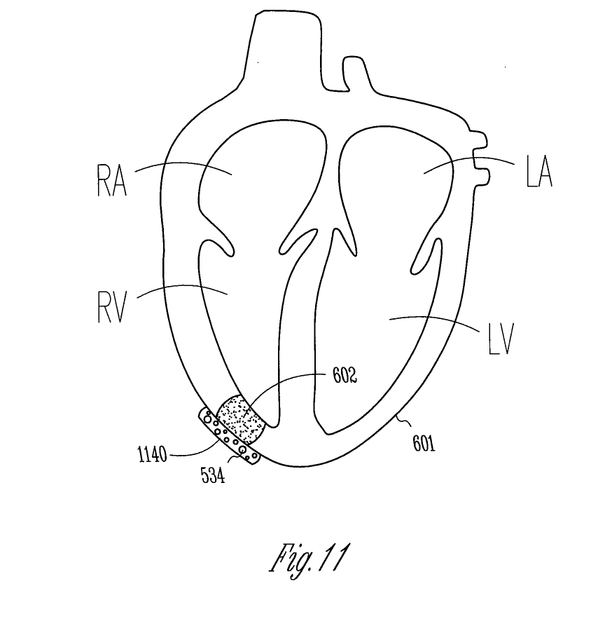

FIG. 11 is an illustration of an embodiment of an epicardial patch 1140, which is another specific embodiment of cardiovascular device 540. Epicardial patch 1140 contains biologic agent 534. In one embodiment, biologic agent 534 is embedded in epicardial patch 1140. In another embodiment, biologic agent 534 is coated on at least a portion of the surface of epicardial patch 1140 that is to be in contact with heart 601.

In one embodiment, epicardial patch 1140 is an extracellular matrix (ECM) scaffold. The ECM scaffold includes ECM isolated from allogeneic or xenogeneic small intestine submucosa, urinary bladder submucosa, and the like, and is applied to injured myocardial region 602 to promote cell regeneration and tissue repair as well as mechanical support in that injured myocardial region. For example, the use of isolated ECM for cardiac repair, such as an isolated ECM support which is applied to cardiac tissue, may result in enhanced transient mechanical support, enhanced angiogenesis, enhanced localization of stem cells, and/or reduced adverse effects, e.g., ventricular remodeling, including reduced fibrosis, of the heart, thereby leading to enhanced function of an infarcted region. An example of an epicardial patch being an ECM scaffold, including the use of ECM in combination with cardiac pacing, is discussed in U.S. patent application Ser. No. 11/017,627, “EPICARDIAL PATCH INCLUDING ISOLATED EXTRACELLULAR MATRIX WITH PACING ELECTRODES,” filed on Dec. 20, 2004, assigned to Cardiac Pacemakers, Inc., which is incorporated herein by reference in its entirety.

In another embodiment, epicardial patch 1140 is an article functioning as a bulking agent applied onto the epicardial surface over injured myocardial region 602 to provide mechanic support. The bulking agent is at least partially made of a polymeric material, in which biologic agent 534 is embedded. In a specific embodiment, biologic agent 534 includes the lyophilized pro-survival or pro-growth factors. In one embodiment, epicardial patch 1140 allows for controllable release of biologic agent 534. In a specific embodiment, the polymeric material is the electrically sensitive polymer discussed above with reference to FIG. 6. The release of biologic agent 534 is controllable by controlling the amplitude of an electrical field, such as using pacing electrodes, if available. In other embodiments, the bulking agent embedded with biologic agent 534 is injected into a portion of the cardiac wall including injured myocardial region 602 or applied onto the endocardial surface over injured myocardial region 602.

The various embodiments of cardiovascular device 540 discussed above may each be used alone or in combination with either other or in combination with other therapies such as cardiac pacing therapy, drug therapy, and other biologic therapy, as determined by a physician. For example, a transvascular device such as device 640, 740, 840, and 940 may be used in combination with stent 1040 and/or epicardial patch 1140. Cardiac remodeling control pacing therapy may be applied in addition to the delivery of biological agent 534.

FIG. 12 is a flow chart illustrating an embodiment of a method 1200 for preparing and delivering pro-growth and/or pro-survival factors to treat a cardiac injury. In one embodiment, method 1200 is performed using biologic agent preparation system 210 and cardiovascular device 540, including their various embodiments as discussed above.

Cells such as fibroblast cells are disposed in growth media at 1210. The cells are of a type that is known to produce the pro-growth and/or pro-survival factors, which have pro-growth and/or pro-survival and optionally differentiation or engraftment effect(s) on cells in vivo resulting in healing of injured tissue. In one embodiment, the fibroblast cells include autologous fibroblast cells. In another embodiment, the fibroblast cells include non-autologous fibroblast cells. In another embodiment, the fibroblast cells include fibroblast cells of a species different from the one receiving the pro-growth and/or pro-survival factors.

A light is applied onto the cells in the growth media at 1220. The light induces the cells to produce pro-growth and/or pro-survival factors, which are released into the growth media. In one embodiment, the light includes one or more components each having a wavelength of approximately 400 nm to 1000 nm and an intensity to approximately 1000 mcd to 10000 mcd, or in combination approximately 0.1 to 50 mW/cm². In one embodiment, light includes one or more components which emit light for a time to cells so as to deliver from about 0.1 to about 10 joules, with 3 joules being a specific example. In a specific embodiment, the light includes a red light having a wavelength of approximately 600 nm to 720 nm, with approximately 630 nm being a specific example. In another specific embodiment, the light includes an infrared light having a wavelength of approximately 720 nm to 1000 nm, with approximately 880 nm being a specific example. In one embodiment, the light is applied using one or more LEDs. In a specific embodiment, the light is applied using one or more LEDs coupled to one or more optical filters each allowing for passage of light within a specified range of wavelength. In one embodiment, the light is applied using a plurality of LEDs emitting lights having substantially different wavelengths. In a specific embodiment, the plurality of LEDs includes at least an infrared LED emitting an infrared light and a red LED emitting a red light.

The pro-growth factors and/or pro-survival factors are collected (isolated) at 1230, by separating the pro-growth factor and/or pro-survival factors in the growth media from the first type of cells. In one embodiment, the cells such as fibroblast cells are first separated, such as by filtering. When necessary or desirable, the resulting filtered fraction is subjected to further separations steps. In another embodiment, for nonadherent cells, cells may be separated from media by centrifugation.

The isolated pro-growth factors and/or pro-survival factors are delivered to an injured region at 1240. In one embodiment, a dry form of the pro-growth factors and/or pro-survival factors is prepared for storage and/or delivery. The pro-growth factors and/or pro-survival factors are lyophilized. In a specific embodiment, the lyophilized pro-growth factors and/or pro-survival factors are embedded in a polymeric material that is coated over at least a portion of a cardiac or transvascular device, such as an implantable transvascular lead, a percutaneous transvascular catheter, a stent, or an epicardial patch. After implantation or catheterization, the pro-growth factors and/or pro-survival factors embedded in the coating are released to an injured myocardial region and/or another location in the cardiovascular system that is upstream from the injured myocardial region. In another embodiment, a liquid form of the pro-growth factors and/or pro-survival factors is prepared for storage and/or delivery. In a specific embodiment, the liquid form is prepared by reconstituting the lyophilized pro-growth factors and/or pro-survival factors. The pro-growth factors and/or pro-survival factors in liquid form are injected into the injured myocardial region and/or another location in the cardiovascular system that is upstream from the injured myocardial region, through a hollow needle or a lumen extending within a transvascular device such as an implantable transvascular lead, or a percutaneous transvascular catheter.

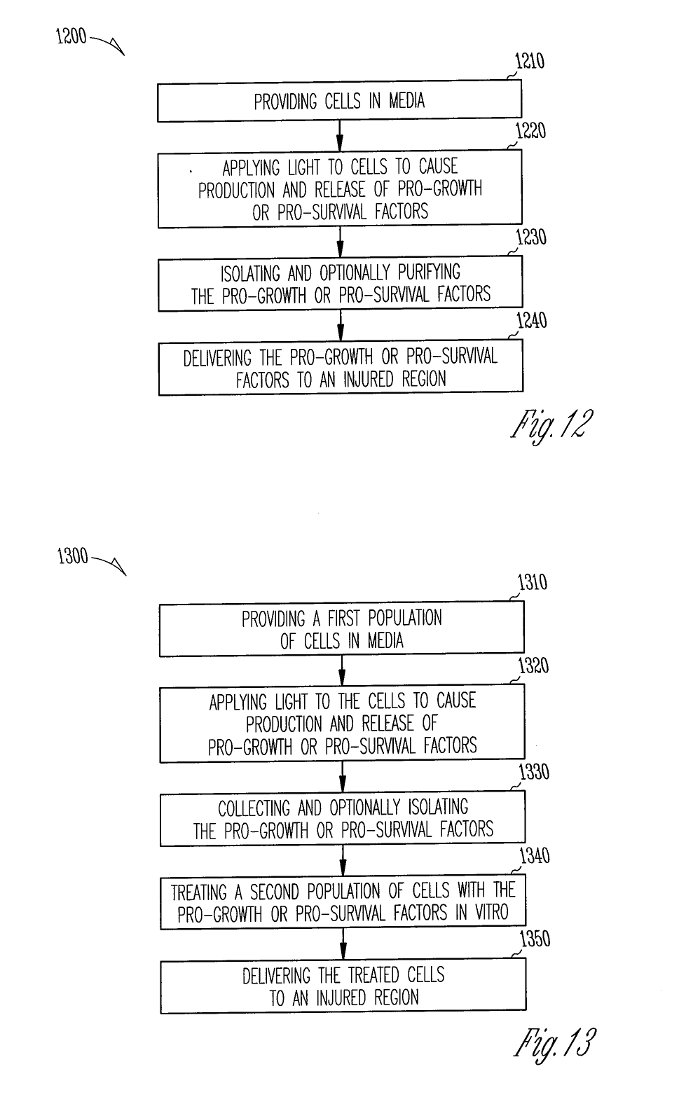

FIG. 13 is a flow chart illustrating an embodiment of a method 1300 for preparing and delivering cells to treat the cardiac injury. In one embodiment, method 1300 is performed using biologic agent preparation system 210 and cardiovascular device 540, including their various embodiments as discussed above.

Step 1310 is substantially identical to step 1210 discussed above with respect to method 1200. Step 1320 is substantially identical to step 1220 discussed above with respect to method 1200.

The pro-growth factors and/or pro-survival factors are isolated at 1330. In one embodiment, the cells such as fibroblast cells are separated from the pro-growth factors and/or pro-survival factors in the growth media. In this embodiment, the isolated pro-growth factors and/or pro-survival factors include the pro-growth factors and/or pro-survival factors and the growth media. In another embodiment, further separation steps are conducted to yield purified pro-growth factors and/or pro-survival factors.

Cells such as stem cells, cardiac cells, or endothelial cells are treated with the isolated pro-growth factors and/or pro-survival factors in vitro at 1340. In one embodiment, the stem cells, cardiac cells, or endothelial cells include autologous cells. In another embodiment, the stem cells, cardiac cells, or endothelial cells include non-autologous cells. The treatment enhances the survival, growth and engraftment, and optionally differentiation of the treated cells.

The treated cells are delivered (i.e., transplanted) to the injured region at 1350. The survival, growth and engraftment and optionally differentiation of the treated cells provides for healing of the injured region. In one embodiment, the pro-growth factors and/or pro-survival factors are also delivered to the injured region to further aid the healing process.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A method for preparing a composition comprising one or more pro-growth or pro-survival factors, comprising:
providing one or more light sources and a first population of mammalian cells in media;
exposing the first population of mammalian cells to an amount of a wavelength of light or a band of wavelengths of light for a time so as to increase the amount of one or more pro-growth factors and/or one or more pro-survival factors in the media, yielding a composition comprising the one or more pro-growth factors and/or one or more pro-survival factors; and
isolating the composition.

2. The method of claim 1, wherein isolating includes separating the media from the cells.

3. The method of claim 1, further comprising purifying the one or more pro-growth and/or pro-survival factors from the isolated composition to yield a purified composition.

4. The method of claim 3, further comprising lyophilizing the purified composition.

5. The method of claim 4, further comprising embedding the lyophilized composition in a polymeric material.

6. The method of claim 3, wherein purifying includes filtering.

7. The method of claim 1, wherein the one or more light sources emit light of 600 nm to 720 nm.

8. The method of claim 1, wherein the one or more light sources emit light of 720 nm to 1000 nm.

9. The method of claim 1, wherein one of the light sources is a light-emitting diode (LED) or an array of LEDs.

10. The method of claim 9, wherein the light sources include a plurality of LEDs and each emit light of a different wavelength or a different band of wavelengths.

11. The method of claim 1, wherein the first population comprises fibroblast cells.

12. The method of claim 1, further comprising contacting a second population of cells with the isolated composition.

13. The method of claim 12, wherein the second population of cells includes stem cells, cardiac cells or endothelial cells.

14. The method of claim 1, further comprising lyophilizing the isolated composition.

15. The method of claim 14, further comprising embedding the lyophilized composition in a polymeric material.

16. The method of claim 15, further comprising applying the polymeric material containing the lyophilized composition to at least a portion of a cardiac device or a transvascular device.

17. The method of claim 4 or 14, further comprising combining a pharmaceutically acceptable carrier with the lyophilized composition.

18. The method of claim 13, further comprising isolating the second population of cells.

19. The method of claim 18, further comprising introducing the isolated second population of cells to a device.

20. The method of claim 18, further comprising introducing the isolated second population of cells to a polymer material.

21. An isolated composition produced by the method of claim 1.

22. A purified composition produced by the method of claim 3.

23. The composition of claim 21 or 22, which is lyophilized.

24. The composition of claim 23, further comprising a pharmaceutically acceptable carrier.

25. A method to identify a wavelength of light that enhances expression of pro-growth factors and/or pro-survival factors comprising:
a) contacting in vitro a first population of mammalian cells in culture media with a wavelength of light or a band of wavelengths of light;
b) collecting the media and contacting the media with a second population of mammalian cells which is different than the first population of cells; and
c) identifying whether the media enhances survival or growth of the second population of cells.

26. The method of claim 25, wherein the first population is contacted with red light.

27. The method of claim 25, wherein the first population is contacted with infrared light.

28. The method of claim 25, wherein the second population of cells comprises bone marrow cells.

29. The method of claim 25, wherein the second population of cells comprises cardiac cells.

30. The method of claim 25, wherein the second population of cells comprises endothelial cells.

31. The method of claim 25, wherein the first population of cells comprises fibroblast cells.

32. The method of claim 25, wherein the first population of cells is from a different species than the second population of cells.

33. The method of claim 32, wherein the second population of cells comprises human cells.

34. The method of claim 1 or 25 wherein the cells are exposed to about 1000 to about 10,000 mcd or about 0.1 to about 50 mW/cm².

35. The method of claim 1 or 25 wherein the cells are exposed to about 0.1 to about 10 joules.
